(19) United States
(12) Patent Application Publication
(10) Pub. No.: US 2021/0074923 A1
(43) Pub. Date: Mar. 11, 2021

(54) ORGANIC ELECTROLUMINESCENT COMPOUND AND ORGANIC ELECTROLUMINESCENT DEVICE CONTAINING THE SAME

(71) Applicant: INT TECH (HK) CO., LIMITED, Central (HK)

(72) Inventor: MIN-HSIEN CHEN, PINGTUNG COUNTY (TW)

(21) Appl. No.: 16/563,476

(22) Filed: Sep. 6, 2019

(51) Int. Cl. H01L 51/00; C07D 241/38; C09K 11/06

(52) U.S. Cl. CPC H01L 51/0061; H01L 51/5016; C09K 11/06; C07D 241/38

(57) ABSTRACT

The present disclosure provides an organic compound represented by the following Formula (I):

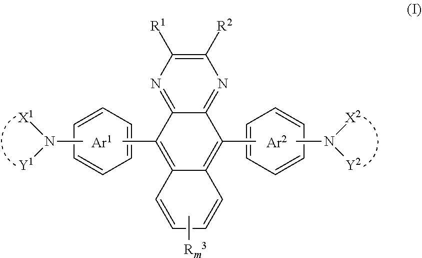

wherein each of R¹ and R² is independently selected from the groups consisting of CN, CF₃ and a substituted or non-substituted ester group, R³ is selected from the groups consisting of hydrogen, a substituted or non-substituted C₁-C₁₂ alkyl group and a substituted or non-substituted C₁-C₁₂ aryl group, and m is an integer between 1 and 4; each of Ar¹ and Ar² is independently selected from the group consisting of an ortho, meta or para substituted C₆ aromatic ring; and each of

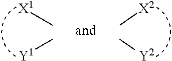

is independently an electron-donating group.

### TECHNICAL FIELD

The present disclosure is related to an electroluminescent compound and an electroluminescent device containing the same, and especially to an organic electroluminescent compound and an organic electroluminescent device containing the same.

### BACKGROUND

Due to great potential in application to display devices, organic light emitting diodes (OLEDs) have recently become very important to the scientific community and the display industry, and now attract much focus in research and development. An OLED is a light-emitting diode (LED) in which a film of organic electroluminescent compounds, placed between an anode and a cathode, emits light in response to excitation such as by an electric current. OLEDs are useful in displays such as television screens, computer monitors, mobile phones, and tablets. OLED devices are self-luminous devices, and have been actively studied for their brightness, superior visibility, and the ability to display clearer images in comparison with liquid crystal devices.

A known example of such an OLED device is one that emits a long-wavelength light of more than 700 nm (such as a near-infrared light). There is currently demand for design and development of novel organic electroluminescent compounds that can emit light having wavelengths greater than 700 nm, so as to provide an OLED device generating such wavelengths with high light-emission efficiency and long service life.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion.

FIG. 1 is a schematic cross-sectional view illustrating an electroluminescent element of an OLED in accordance with some embodiments of the present disclosure.

FIG. 2 is a schematic cross-sectional view illustrating an organic electroluminescent device in accordance with some embodiments of the present disclosure.

FIG. 3 is a schematic cross-sectional view illustrating an organic electroluminescent device in accordance with some embodiments of the present disclosure.

### DETAILED DESCRIPTION OF THE DISCLOSURE

The following disclosure provides many different embodiments, or examples, for implementing different features of the provided subject matter. Specific examples of components and arrangements are described below to simplify the present disclosure. These are, of course, merely examples and are not intended to be limiting.

The organic electroluminescent compound works on the principal that electrons and holes diffuse through an electron transportation layer and hole transportation layer, respectively, to enter a light-emitting layer, and recombine in the emitting region to form a particle generally referred to as an exciton. When an external voltage is applied to an OLED device, electrons and holes are injected from a cathode and an anode, respectively. Electrons are injected from a cathode into a lowest unoccupied molecular orbital (LUMO) and holes are injected from an anode into a highest occupied molecular orbital (HOMO). When the electrons recombine with holes in the EML, excitons are formed and the excitons then emit light.

A known example of such an organic electroluminescent compound is one that emits a light having a wavelength of greater than 700 nm. The present disclosure therefore provides an organic electroluminescent compound represented by the following Formula (I):

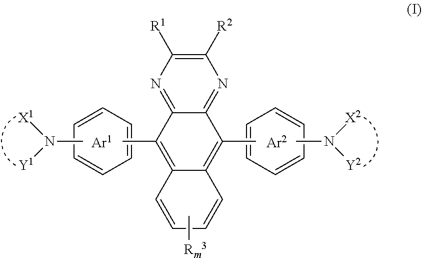

wherein

each of R¹ and R² is independently selected from the groups consisting of CN, CF₃ and a substituted or non-substituted ester group,

R³ is selected from the groups consisting of hydrogen, a substituted or non-substituted C₁-C₁₂ alkyl group and a substituted or non-substituted C₁-C₁₂ aryl group, and m is an integer between 1 and 4;

each of Ar¹ and Ar² is independently selected from the group consisting of an ortho, meta or para substituted C₆ aromatic ring; and

each of

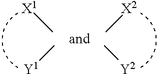

is independently an electron-donating group.

In some embodiments, the organic electroluminescent compound represented by Formula (I) emits a light having a wavelength of more than 700 nm under a bias voltage. In some embodiments, the organic electroluminescent compound represented by Formula (I) emits a near-IR light under a bias voltage.

In some embodiments, each of R¹ and R² is independently selected from the groups consisting of CN and CF₃.

In some embodiments, each of R³ is selected from the groups consisting of hydrogen and a substituted or non-substituted C₁-C₆ alkyl group, and m is an integer between 1 and 4. In some embodiments, each of R³ is hydrogen.

In some embodiments, each of Ar¹ and Ar² is independently a para substituted C₆ aromatic ring.

In some embodiments, each of

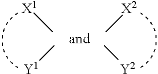

is independently selected from the group consisting of the electron-donating groups represented by formula (i), formula (ii), formula (iii), formula (iv), formula (v), formula (vi) and formula (vii):

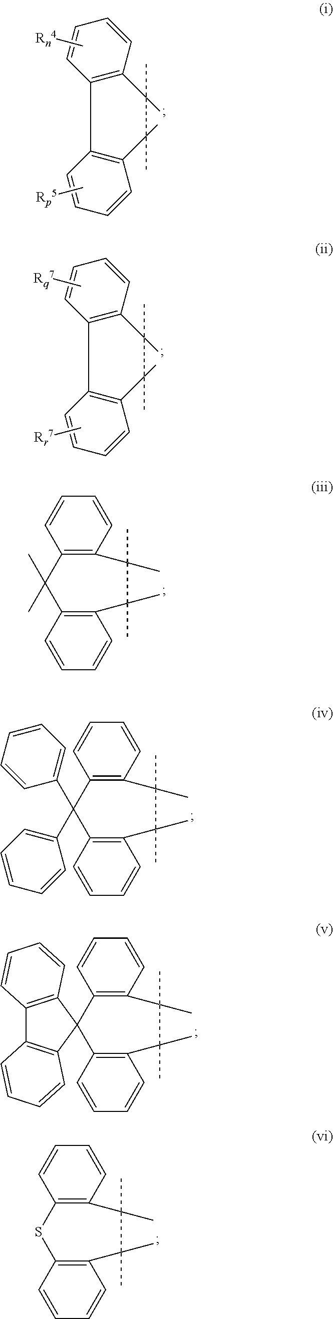

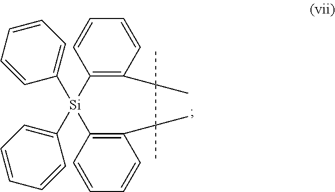

wherein

each of R⁴, R⁵, R⁶ and R⁷ is independently selected from the groups consisting of hydrogen, a substituted or non-substituted C₁-C₁₂ alkyl group, a substituted or non-substituted C₁-C₁₂ aryl group and a substituted or non-substituted C₁-C₁₂ alkoxy group, and

each of n, p, q and r is independently an integer between 1 and 4.

In some embodiments, each of

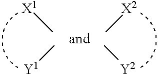

is independently selected from the group consisting of the electron-donating groups represented by formula (i), formula (ii) and formula (iii).

In some embodiments, substituents of the substituted groups in R⁴, R⁵, R⁶ and R⁷ each independently includes at least one selected from the group consisting of deuterium, a halogen, a (C₁-C₃₀)alkyl group, a (C₁-C₃₀)alkyl group substituted with a halogen, a (C₆-C₃₀)aryl group, a 3- to 30-membered heteroaryl group, a 3- to 30-membered heteroaryl group substituted with a (C₆-C₃₀)aryl group, a (C₆-C₃₀)aryl group substituted with a 3- to 30-membered heteroaryl group, a (C₃-C₃₀)cycloalkyl group, a 5- to 7-membered heterocycloalkyl group, a tri(C1-C30)alkylsilyl group, a tri(C₆-C₃₀)arylsilyl group, a di(C₁-C₃₀)alkyl(C₆-C₃₀)arylsilyl group, a (C₁-C₃₀)alkyldi(C₆-C₃₀)arylsilyl group, a (C₂-C₃₀)alkenyl group, a (C₂-C₃₀)alkynyl group, a cyano group, a di(C₁-C₃₀)alkylamino group, a di(C₆-C₃₀)arylamino group, a (C₁-C₃₀)alkyl(C₆-C₃₀)arylamino group, a di(C6-C30)arylboronyl group, a di(C₁-C₃₀)alkylboronyl group, a (C₁-C₃₀)alkyl(C₆-C₃₀)arylboronyl group, a (C₆-C₃₀)aryl(C₁-C₃₀)alkyl group, a (C₁-C₃₀)alkyl(C₆-C₃₀)aryl group, a carboxyl group, a nitro group and a hydroxyl group. [to client: please check]

In some embodiments of the present disclosure, the terms “alkyl” and “alkoxy,” and any alkyl moiety that is comprised in substituents, include both a linear structure and a branched structure, and the term “cycloalkyl” includes a mono- or polycyclic hydrocarbon or a substituted or unsubstituted (C₇-C₃₀) bicycloalkyl group. The term “aryl” refers to an organic radical that: is derived from an aromatic hydrocarbon by removing one hydrogen atom; includes a monocyclic ring or fused ring each of whose rings has 4 to 7, preferably 5 or 6, ring backbone atoms; may be formed by linking two or more aryl groups to one another via one or more single bonds; and includes phenyl, biphenyl, terphenyl, naphthyl, anthryl, indenyl, fluorenyl, phenanthrenyl, triphenylenyl, pyrenyl, perylenyl, chrysenyl, naphthacenyl, fluoranthenyl, etc., wherein said naphthyl includes 1-naphthyl and 2-naphthyl, said anthryl includes 1-anthryl, 2-anthryl and 9-anthryl, and said fluorenyl includes 1-fluorenyl, 2-fluorenyl, 3-fluorenyl, 4-fluorenyl and 9-fluorenyl. The term “heteroaryl” refers to an aryl that: has 1 to 4 heteroatoms selected from the group consisting of B, N, O, S, P(═O), Si and P, and carbon atoms as remaining ring backbone atoms other than said heteroatom; is a monocyclic ring or fused ring condensed with at least one benzene ring; may be partially saturated; may be formed by linking at least one heteroaryl group to another heteroaryl or aryl group via one or more single bonds; may be a divalent aryl group whose ring backbone heteroatom is oxidized or quaternarized, for example, to form an N-oxide or a quaternary salt; and includes a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., a fused ring-type heteroaryl including benzofuranyl, benzothiophenyl, isobenzofuranyl, benzoimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl quinazolinyl, quinoxalinyl, carbazolyl, phenanthridinyl, benzodioxolyl, dibenzofuranyl, dibenzothiophenyl, etc., N-oxides thereof (for example, pyridyl N-oxide, quinolyl N-oxide), and quaternary salts thereof.

In some embodiments, the organic compound is represented by the following Formula (II):

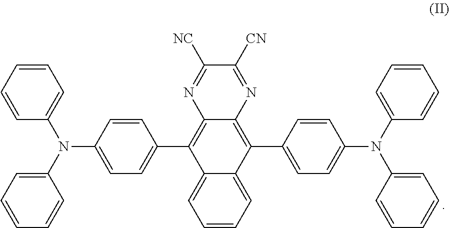 [SMILES: N#Cc1nc2c(-c3ccc(N(c4ccccc4)c4ccccc4)cc3)c3ccccc3c(-c3ccc(N(c4ccccc4)c4ccccc4)cc3)c2nc1C#N]

In some embodiments, the organic electroluminescent compound represented by Formula (II) emits a light having a wavelength of more than 700 nm under a bias voltage. In some embodiments, the organic electroluminescent compound represented by Formula (II) emits a near-IR light under a bias voltage.

In some embodiments, the present invention provides a method of preparing the organic electroluminescent compound represented by the Formula (I). In some embodiments, the present invention provides a method of preparing the main body of organic electroluminescent compound represented by the Formula (I). In some embodiments, the present invention provides a method of preparing the main body of organic electroluminescent compound represented by the Formula (II). In some embodiments, the organic electroluminescent compound represented by Formula (II) can be synthesized according to Scheme 1:

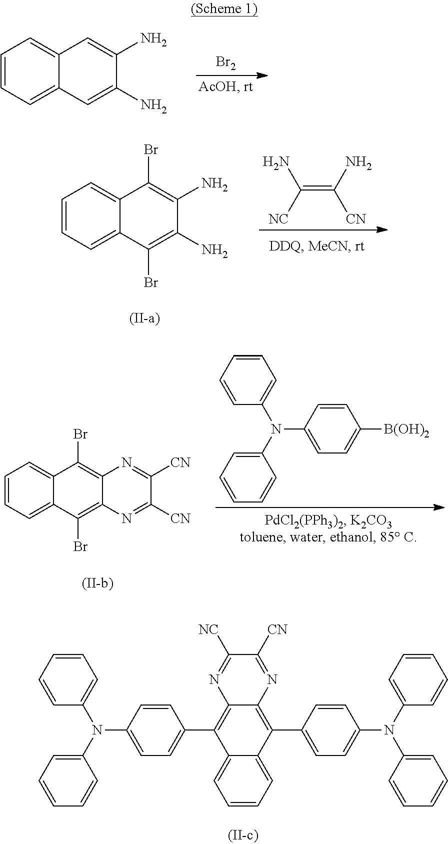

In Scheme 1, the organic electroluminescent compound represented by Formula (II) is synthesized by three steps, the first step, the second step, and the third step.

In the first step, the compound represented by Formula (II-a) was synthesized as follows. 1.58 g 2,3-diaminonaphthalene and 25 mL acetic acid were added into a flask. A bromine/acetic acid solution (1.14 mL Br₂ in 10 mL acetic acid) was further added dropwise into the flask at room temperature and stirred for 2 to 3 hours to obtain a reaction mixture. The reaction mixture was filtered to obtain a precipitate. The precipitate was washed with aqueous K₂CO₃ solution and water to obtain the compound represented by Formula (II-a).

In the second step, the compound represented by Formula (II-b) was synthesized as follows. 0.59 g diaminomaleonitrile (5.5 mmol) and 20 mL acetonitrile were added into a round-bottom bottle at room temperature to obtain a dark brown solution. 1.25 g 2,3-Dichloro-5,6-dicyano-1,4-benzoquinone was further added portionwise into the round-bottom bottle and stirred for 20 minutes to obtain a light brown solution. The light brown solution was filtered to obtain a light brown solid matter. The light brown solid matter was then mixed with 1.58 g 1,4-dibromo-2,3-diamino naphthalene(5.0 mmol) to obtain a mixed solid. The mixed solid was then added portionwise into 30 mL trifluoroacetic acid to obtain a mixture. After the mixture was stirred overnight, the mixture was diluted with water, and the precipitate of the mixture was collected by filtration and washed with acetonitrile. The precipitate was further purified by column chromatography (DCM/Hex=1/1) to obtain the compound represented by Formula (II-b).

In the third step, the compound represented by Formula (II-c) was synthesized as follows. 0.39 g (1.0 mmol) 5,10-dibromobenzo[g]quinoxaline-2,3-dicarbonitrile, 0.35 g (1.2 mmol) (4-(diphenylamino)phenyl)boronic acid, 0.096 g PdCl₂(PPh₃)₂, and 0.42 g (3.0 mmol) potassium dicarbonate were added in a round bottom bottle under a nitrogen gas atmosphere. 6 mL solvent of toluene/water/ethanol (3/2/1) was further added into the round bottom bottle to obtain a mixture. The mixture was heated to 90° C. and stirred for 12 hours. The mixture was then cooled to room temperature and extracted with EA, and further washed with brine. The organic layer extracted from the mixture was collected and dried by magnesium sulfate. The organic solvent of the organic layer was evaporated and the crude of the organic layer was further purified by column chromatography (Hex/EA=9/1) to obtain the compound represented by Formula (II-c).

In the present disclosure, an OLED is disclosed. The OLED includes a pair of electrodes including an anode and a cathode, and an electroluminescent element disposed between the pair of electrodes, wherein the electroluminescent element comprises the aforementioned organic electroluminescent compound represented by Formula (I). The details of the compound have been recited previously and will not be repeated herein. In some embodiments, the organic electroluminescent compound represented by the Formula (I) of the invention is useful as a light-emitting material of the OLED. Accordingly, the organic electroluminescent compound represented by the Formula (I) of the invention may be effectively used as a light-emitting material in an electroluminescent element of an OLED. In some embodiments, the electroluminescent element comprises the aforementioned organic electroluminescent compound represented by Formula (II). The details of the compounds have been recited previously and will not be repeated herein.

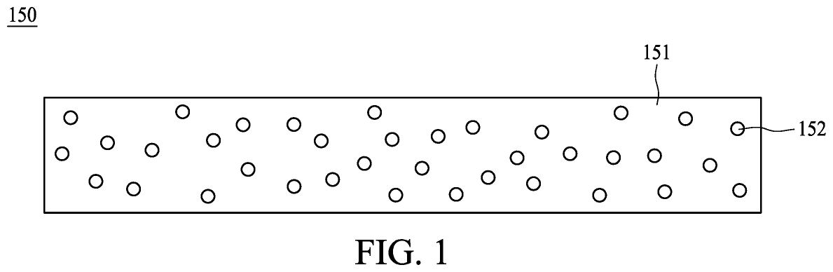

FIG. 1 is a schematic cross-sectional view illustrating an electroluminescent element of an OLED according to aspects of the present disclosure in some embodiments. In some embodiments, the electroluminescent element is an emitting layer 150. In some embodiments, the emitting layer 150 emits light having a wavelength greater than 700 nm under a bias voltage. Thus, the present disclosure provides an invention relating to a near-IR light emitter having the structure represented by the Formula (I), and an invention relating to the use of the compound as the near-IR light emitter. In some embodiments, the OLED that uses the compound as a light-emitting material can emit near-IR light while providing high light-emission efficiency and long service life.

In some embodiments, as shown in FIG. 1, the emitting layer 150 is made of host emitting material 151 doped with a suitable light-emitting material. In some embodiments, the emitting layer 150 includes a host emitting material 151 and a dopant 152.

In some embodiments, the dopant 152 is the aforementioned organic electroluminescent compound represented by the Formula (I). In some embodiments, the dopant 152 is the aforementioned organic electroluminescent compound represented by the Formula (II). Thus, the OLED including the light-emitting layer 150 features advantages of a practical light-emission efficiency and long service life. By adding the dopant 152 in the emitting layer 150, the energy of the host emitting material 151 can be transferred to the dopant 152, so that the light color and the luminous efficiency of the host emitting material 151 can be changed, thus broadening the application of the OLED.

In some embodiments, the host emitting material 151 is the aforementioned organic electroluminescent compound represented by the Formula (I). In some embodiments, the host emitting material 151 is the aforementioned organic electroluminescent compound represented by the Formula (II).

In the present disclosure, an organic electroluminescent device is disclosed. The organic electroluminescent device includes a pair of electrodes, and an electroluminescent element disposed between the pair of electrodes comprising an organic electroluminescent compound represented by Formula (I). The details of the compound have been recited previously and will not be repeated herein. In some embodiments, the electroluminescent element disposed between the pair of electrodes comprises the aforementioned organic electroluminescent compound represented by the Formula (II). In some embodiments, the electroluminescent element is an organic layer. In some embodiments, the pair of electrodes includes cathode and anode. In some embodiments, the organic electroluminescent device 100 further includes carrier transportation layers and carrier injection layers disposed between the electrodes.

In some embodiments, a plurality of organic layers are further disposed between the pair of electrodes. In some embodiments, the electroluminescent element is disposed between a first type carrier transportation layer and a second type carrier transportation layer. The first type is opposite to the second type. In some embodiments, the first type transportation layer is a hole transportation layer (HTL) and the second type carrier transportation layer is an electron transportation layer (ETL). In some embodiments, the first type transportation layer is an electron transportation layer and the second type carrier transportation layer is a hole transportation layer.

In some embodiments, the first type carrier transportation layer is a composite structure and includes at least a primary layer and a secondary transportation layer. Similarly, in some embodiments, the second type carrier transportation layer is a composite structure and includes at least a primary layer and a secondary transportation layer.

In some embodiments, the organic electroluminescent device further includes a first type carrier injection layer adjacent to the first type carrier transportation layer. In some embodiments, the first type carrier injection layer is disposed between the first type carrier transportation layer and one of the electrodes. Similarly, the organic electroluminescent device further includes a second type carrier injection layer adjacent to the second type carrier transportation layer. In some embodiments, the second type carrier injection layer is disposed between the second type carrier transportation layer and the other one of the electrodes.

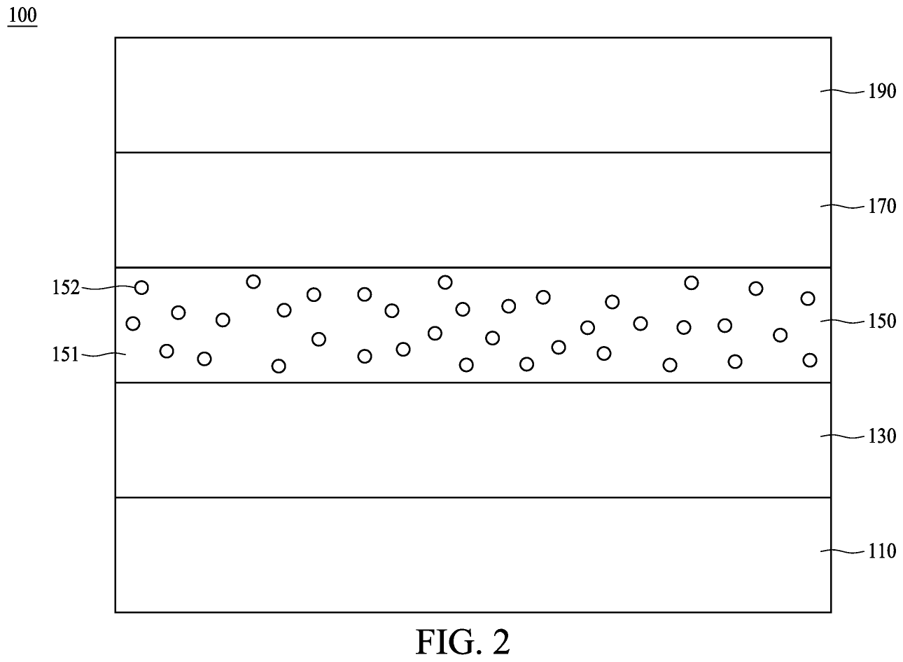

FIG. 2 is a schematic cross-sectional view illustrating an organic electroluminescent device according to aspects of the present disclosure in some embodiments. The configuration of the organic electroluminescent device of the present invention is not particularly limited. In some embodiments, as shown in FIG. 2, the organic electroluminescent device 100 includes an anode 110, a hole transportation layer 130, an emitting layer 150, an electron transportation layer 170 and a cathode 190. The details of the emitting layer 150 have been recited previously and will not be repeated herein.

In some embodiments, the anode 110 can be obtained from a conductor having high work function to facilitate the injection of holes in the emitting layer 150. The material of the anode 110 is, for instance, metal, metal oxide, a conducting polymer, or a combination thereof. In some embodiments, the metal is, for instance, Ni, Pt, V, Cr, Cu, Zn, Au, or an alloy thereof; the metal oxide is, for instance, zinc oxide, indium oxide, indium tin oxide (ITO), or indium zinc oxide (IZO); the combination of the metal and the oxide is, for instance, a combination of ZnO and Al or a combination of SnO₂ and Sb; and the conductive polymer is, for instance, poly(3-methylthiophene), poly(3,4-(ethylene-1,2-dioxy)thiophene (PEDT), polypyrrole, or polyaniline, but the invention is not limited thereto.

In some embodiments, the cathode 190 can be obtained from a conductor having low work function to facilitate the injection of electrons in the light-emitting layer 150. In some embodiments, the material of the cathode 190 is, for instance, metal or multilayer structure material. In some embodiments, the metal is, for instance, Mg, Ca, Na, K, Ti, In, Y, Li, Gd, Al, Ag, Sn, Pd, Cs, Ba, or an alloy thereof; the material of the multilayer structure is, for instance, LiF/Al, LiO₂/Al, LiF/Ca, LiF/Al, or BaF₂/Ca, but the invention is not limited thereto.

In some embodiments, the organic layers further include a hole-blocking layer (HBL) between the emitting layer 150 and the electron transportation layer 170 or further include an electron-blocking layer (EBL) between the emitting layer 150 and the hole transportation layer 130. In some embodiments, due to the longer service life and the diffusion length of triplet excitons compared to those of singlet excitons, when the organic electroluminescent device is a phosphorescent organic electroluminescent device, the additional hole-blocking layer and electron-blocking layer are especially needed. The purpose of the use of a hole-blocking layer or an electron-blocking layer is to confine the recombination of injected holes and electrons and the relaxation of created excitons within the emitting layer, thus improving the device's efficiency. To serve such functions, the hole-blocking materials or electron-blocking materials must have HOMO and LUMO energy levels suitable to block holes or electrons from being transported from the emitting layer 150 to the electron transportation layer 170 or the hole transportation layer 130.

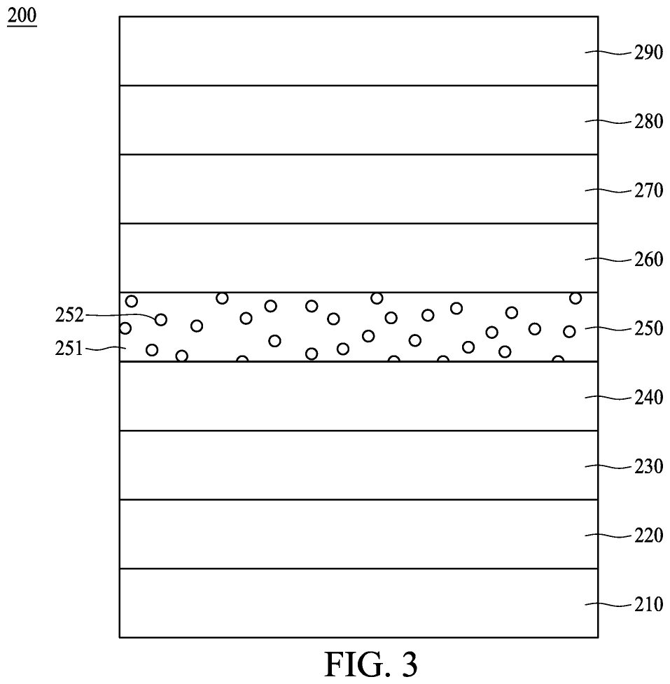

FIG. 3 is a schematic cross-sectional view illustrating an organic electroluminescent device according to aspects of the present disclosure in some embodiments. In some embodiments, as shown in FIG. 3, the organic electroluminescent device 200 includes an anode 210, a hole-injecting layer (HIL) 220, a hole transportation layer 230, an electron-blocking layer 240, an emitting layer 250, a hole-blocking layer 260, an electron transportation layer 270, an electron-injecting layer (EIL) 280, and a cathode 290. In some embodiments, the hole-injecting layer 220, the hole transportation layer 230, the electron-blocking layer 240, the emitting layer 250, the hole-blocking layer 260, the electron transportation layer 270, the electron-injecting layer 280, and the cathode 290 are sequentially configured on top of the anode 210.

In some embodiments, the organic electroluminescent compound represented by Formula (I) is a host emitting material 251 of the emitting layer 250 of the organic electroluminescent device 200. In some embodiments, the organic electroluminescent compound represented by Formula (II) is the host emitting material 251 of the emitting layer 250 of the OLED device 200.

In some embodiments, the organic compound represented by Formula (I) is a dopant 252 of the emitting layer 250 of the OLED device 200. In some embodiments, the organic electroluminescent compound represented by Formula (II) is the dopant 252 of the emitting layer 250 of the OLED device 200.

In some embodiments, the organic electroluminescent compound represented by Formula (I) is a hole transportation material of the hole transportation layer 230 of the organic electroluminescent device 200. In some embodiments, the organic electroluminescent compound represented by Formula (II) is the hole transportation material of the hole transportation layer 230 of the organic electroluminescent device 200.

In some embodiments, the organic electroluminescent compound represented by Formula (I) is an electron transport material of the electron transportation layer 270 of the organic electroluminescent device 200. In some embodiments, the organic electroluminescent compound represented by Formula (II) is the electron transportation material of the electron transportation layer 270 of the organic electroluminescent device 200.

In some embodiments, the organic electroluminescent compound represented by Formula (I) is a hole-blocking material of the hole-blocking layer 260 of the organic electroluminescent device 200. In some embodiments, the organic electroluminescent compound represented by Formula (II) is the hole-blocking material of the hole-blocking layer 260 of the organic electroluminescent device 200.

In some embodiments, the organic electroluminescent compound represented by Formula (I) is an electron-blocking material of the electron-blocking layer 240 of the organic electroluminescent device 200. In some embodiments, the organic electroluminescent compound represented by Formula (II) is the electron-blocking material of the electron-blocking layer 240 of the organic electroluminescent device 200.

In some embodiments, the organic electroluminescent compound represented by Formula (I) is a hole-injecting material of the hole-injecting layer 220 of the organic electroluminescent device 200. In some embodiments, the organic electroluminescent compound represented by Formula (II) is the hole-injecting material of the hole-injecting layer 220 of the organic electroluminescent device 200.

In some embodiments, the organic electroluminescent compound represented by Formula (I) is an electron-injecting material of the electron-injecting layer 280 of the organic electroluminescent device 200. In some embodiments, the organic electroluminescent compound represented by Formula (II) is the electron-injecting material of the electron-injecting layer 280 of the organic electroluminescent device 200.

In some embodiments of the present disclosure, the use of an organic electroluminescent compound represented by the Formulas (I) and (II) in an electroluminescent element can provide an organic electroluminescent device with a high efficiency and long lifetime.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. An organic electroluminescent compound represented by the following Formula (I): 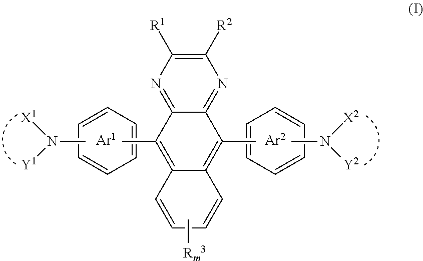
wherein
each of R¹ and R² is independently selected from the groups consisting of CN, CF₃ and a substituted or non-substituted ester group,
R³ is selected from the groups consisting of hydrogen, a substituted or non-substituted C₁-C₁₂ alkyl group and a substituted or non-substituted C₁-C₁₂ aryl group, and m is an integer between 1 and 4;
each of Ar¹ and Ar² is independently selected from the group consisting of an ortho, meta or para substituted C₆ aromatic ring; and
each of 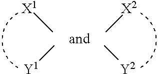
is independently an electron-donating group.

2. The organic electroluminescent compound of claim 1, wherein each of 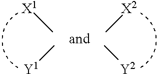
is independently selected from the group consisting of the electron-donating groups represented by formula (i), formula (ii), formula (iii), formula (iv), formula (v), formula (vi) and formula (vii): 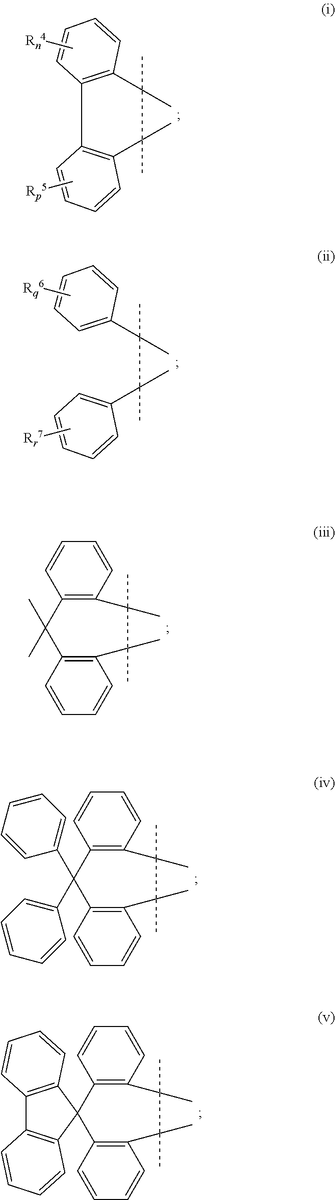 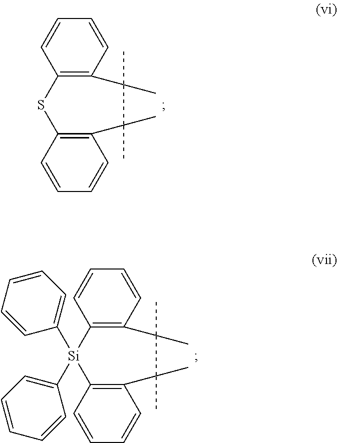
wherein
each of R⁴, R⁵, R⁶ and R⁷ is independently selected from the groups consisting of hydrogen, a substituted or non-substituted C₁-C₁₂ alkyl group, a substituted or non-substituted C₁-C₁₂ aryl group and a substituted or non-substituted C₁-C₁₂ alkoxy group, and
each of n, p, q and r is independently an integer between 1 and 4.

3. The organic electroluminescent compound of claim 2, wherein each of 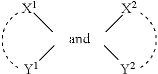
is independently selected from the group consisting of the electron-donating groups represented by formula (i), formula (ii) and formula (iii).

4. The organic electroluminescent compound of claim 1, wherein each of R¹ and R² is independently selected from the groups consisting of CN and CF₃.

5. The organic electroluminescent compound of claim 1, wherein each of R³ is selected from the groups consisting of hydrogen and a substituted or non-substituted C₁-C₆ alkyl group, and m is an integer between 1 and 4.

6. The organic electroluminescent compound of claim 1, wherein each of Ar¹ and Ar² is independently a para substituted C₆ aromatic ring.

7. The organic electroluminescent compound of claim 1, wherein the organic compound is represented by the following Formula (II):

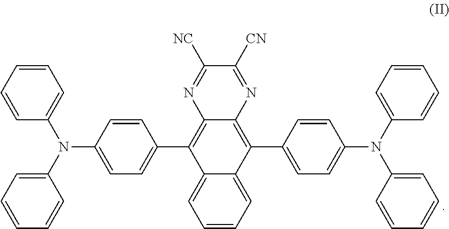 [SMILES: N#Cc1nc2c(-c3ccc(N(c4ccccc4)c4ccccc4)cc3)c3ccccc3c(-c3ccc(N(c4ccccc4)c4ccccc4)cc3)c2nc1C#N]

8. An organic light-emitting device, comprising:
a pair of electrodes; and
an electroluminescent element disposed between the pair of electrodes, wherein the electroluminescent element comprises an organic compound represented by the following Formula (I): 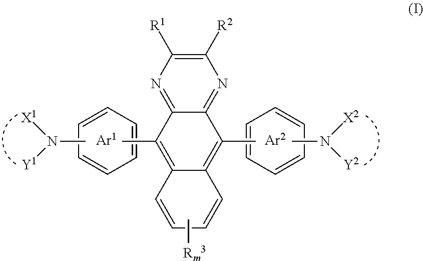
wherein
each of R¹ and R² is independently selected from the groups consisting of CN, CF₃ and a substituted or non-substituted ester group,
R³ is selected from the groups consisting of hydrogen, a substituted or non-substituted C₁-C₁₂ alkyl group and a substituted or non-substituted C₁-C₁₂ aryl group, and in is an integer between 1 and 4;
each of Ar¹ and Ar² is independently selected from the group consisting of an ortho, meta or para substituted C₆ aromatic ring; and
each of 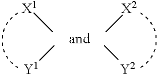
is independently selected from the group consisting of the electron-donating groups represented by formula (i), formula (ii), formula (iii), formula (iv), formula (v), formula (vi) and formula (vii): 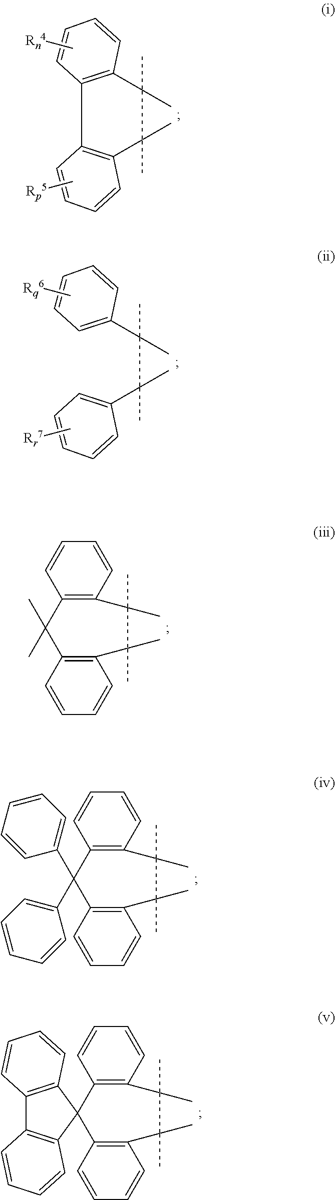 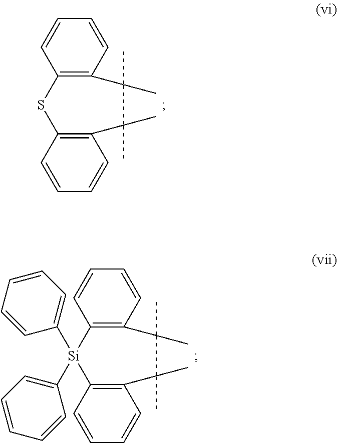
wherein
each of R⁴, R⁵, R⁶ and R⁷ is independently selected from the groups consisting of hydrogen, a substituted or non-substituted C₁-C₁₂ alkyl group, a substituted or non-substituted C₁-C₁₂ aryl group and a substituted or non-substituted C₁-C₁₂ alkoxy group, and
each of n, p, q and r is independently an integer between 1 and 4.

9. The organic light-emitting device according to claim 8, wherein the organic light-emitting device emits a light having a wavelength of more than 700 nm.

10. An organic electroluminescent device, comprising:
a cathode;
an anode; and
an organic layer disposed between the cathode and the anode and comprising an organic compound represented by the following Formula (I): 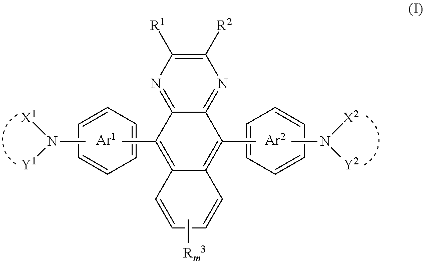
wherein
each of R¹ and R² is independently selected from the groups consisting of CN, CF₃ and a substituted or non-substituted ester group,
R³ is selected from the groups consisting of hydrogen, a substituted or non-substituted C₁-C₁₂ alkyl group and a substituted or non-substituted C₁-C₁₂ aryl group, and m is an integer between 1 and 4;
each of Ar¹ and Ar² is independently selected from the group consisting of an ortho, meta or para substituted C₆ aromatic ring; and
each of 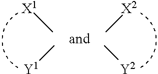
is independently selected from the group consisting of the electron-donating groups represented by formula (i), formula (ii), formula (iii), formula (iv), formula (v), formula (vi) and formula (vii): 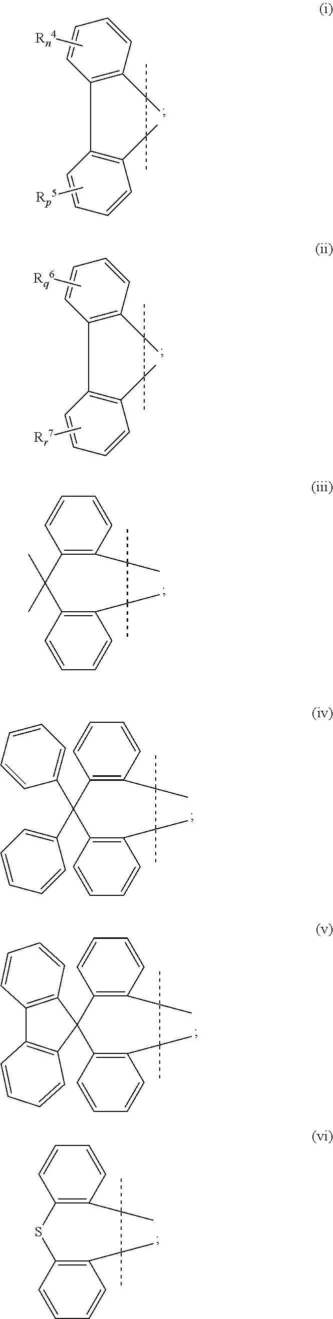 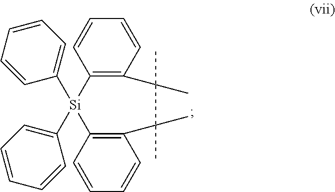
wherein
each of R⁴, R⁵, R⁶ and R⁷ is independently selected from the groups consisting of hydrogen, a substituted or non-substituted C₁-C₁₂ alkyl group, a substituted or non-substituted C₁-C₁₂ aryl group and a substituted or non-substituted C₁-C ₁₂ alkoxy group, and
each of n, p, q and r is independently an integer between 1 and 4.

11. The organic electroluminescent device of claim 10, wherein the organic layer comprises an emitting layer, and the organic compound represented by Formula (I) is a host emitting material.

12. The organic electroluminescent device of claim 10, wherein the organic layer comprises an emitting layer, and the organic compound represented by Formula (I) is a dopant.

13. The organic electroluminescent device of claim 10, wherein the emitting layer emits a near IR light under a bias voltage.

14. The organic electroluminescent device of claim 10, further comprising a light-emitting layer disposed between the cathode and the anode.
